SEQUENCE LISTING (16 sequences)

SEQ ID NO: 1 - DNA, 23 bp, Artificial Sequence
  Description of Artificial Sequence  oligo linker
aattgtcttaattaaccgcttaa

SEQ ID NO: 2 - DNA, 19 bp, Artificial Sequence
  Description of Artificial Sequence oligonucleotide
aattgtcttaattaaccgc

SEQ ID NO: 3 - DNA, 19 bp, Artificial Sequence
  Description of Artificial Sequence oligonucleotide
aattgcggttaattaagac

SEQ ID NO: 4 - DNA, 47 bp, Artificial Sequence
  Description of Artificial Sequence  primer
ctgtacgtaccagtgcactggcctaggcatggaaaaatacataactg

SEQ ID NO: 5 - DNA, 64 bp, Artificial Sequence
  Description of Artificial Sequence  primer
gcggatccttcgaaccatggtaagcttggtaccgctagcgttaaccgggcgactcagtcaatcg

SEQ ID NO: 6 - DNA, 28 bp, Artificial Sequence
  Description of Artificial Sequence  primer
gcgccaccatgggcagagcgatggtggc

SEQ ID NO: 7 - DNA, 50 bp, Artificial Sequence
  Description of Artificial Sequence  primer
gttagatctaagcttgtcgacatcgatctactaacagtagagatgtagaa

SEQ ID NO: 8 - DNA, 10 bp, Artificial Sequence
  Description of Artificial Sequence oligonucleotide
ttaagtcgac

SEQ ID NO: 9 - DNA, 67 bp, Artificial Sequence
  Description of Artificial Sequence oligonucleotide
gccatccctaggaagcttggtaccggtgaattcgctagcgttaacggatcctctagacgagatctgg

SEQ ID NO: 10 - DNA, 67 bp, Artificial Sequence
  Description of Artificial Sequence oligonucleotide
ccagatctcgtctagaggatccgttaacgctagcgaattcaccggtaccaagcttcctagggatggc

SEQ ID NO: 11 - DNA, 39 bp, Artificial Sequence
  Description of Artificial Sequence  primer
gatcggtaccactgcagtggtcaatattggccattagcc

SEQ ID NO: 12 - DNA, 29 bp, Artificial Sequence
  Description of Artificial Sequence  primer
gatcaagcttccaatgcaccgttcccggc

SEQ ID NO: 13 - protein, 1153 aa, Homo sapiens [sequence omitted]

SEQ ID NO: 14 - protein, 1433 aa, Homo sapiens [sequence omitted]

SEQ ID NO: 15 - protein, 1203 aa, Homo sapiens
  CHAIN (1)..(1203): Human nitric oxide synthase
MGNLKSVAQEPGPPCGLGLGLGLGLCGKQGPATPAPEPSRAPASLLPPAPEHSPPSSPLTQPPEGPKFPRVKNWEVGSITYDTLSAQAQQDGPCTPRRCLGSLVFPRKLQGRPSPGPPAPEQLLSQARDFINQYYSSIKRSGSQAHEQRLQEVEAEVAATGTYQLRESELVFGAKQAWRNAPRCVGRIQWGKLQVFDARDCRSAQEMFTYICNHIKYATNRGNLRSAITVFPQRCPGRGDFRIWNSQLVRYAGYRQQDGSVRGDPANVEITELCIQHGWTPGNGRFDVLPLLLQAPDEPPELFLLPPELVLEVPLEHPTLEWFAALGLRWYALPAVSNMLLEIGGLEFPAAPFSGWYMSTEIGTRNLCDPHRYNILEDVAVCMDLDTRTTSSLWKDKAAVEINVAVLHSYQLAKVTIVDHHAATASFMKHLENEQKARGGCPADWAWIVPPISGSLTPVFHQEMVNYFLSPAFRYQPDPWKGSAAKGTGITRKKTFKEVANAVKISASLMGTVMAKRVKATILYGSETGRAQSYAQQLGRLFRKAFDPRVLCMDEYDVVSLEHETLVLVVTSTFGNGDPPENGESFAAALMEMSGPYNSSPRPEQHKSYKIRFNSISCSDPLVSSWRRKRKESSNTDSAGALGTLRFCVFGLGSRAYPHFCAFARAVDTRLEELGGERLLQLGQGDELCGQEEAFRGWAQAAFQAACETFCVGEDAKAAARDIFSPKRSWKRQRYRLSAQAEGLQLLPGLIHVHRRKMFQATIRSVENLQSSKSTRATILVRLDTGGQEGLQYQPGDHIGVCPPNRPGLVEALLSRVEDPPAPTEPVAVEQLEKGSPGGPPPGWVRDPRLPPCTLRQALTFFLDITSPPSPQLLRLLSTLAEEPREQQELEALSQDPRRYEEWKWFRCPTLLEVLEQFPSVALPAPLLLTQLPLLQPRYYSVSSAPSTHPGEIHLTVAVLAYRTQDGLGPLHYGVCSTWLSQLKPGDPVPCFIRGAPSFRLPPDPSLPCILVGPGTGIAPFRGFWQERLHDIESKGLQPTPMTLVFGCRCSQLDHLYRDEVQNAQQRGVFGRVLTAFSREPDNPKTYVQDILRTELAAEVHRVLCLERGHMFVCGDVTMATNVLQTVQRILATEGDMELDEAGDVIGVLRDQQRYHEDIFGLTLRTQEVTSRIRTQSFSLQERQLRGAVPWAFDPPGSDTNSP

SEQ ID NO: 16 - DNA, 240 bp, Homo sapiens
  misc_feature (1)..(240): nucleotides coding for human nitric oxide synthase
gaattcccactctgctgcctgctccagcagacggacgcacagtaacatgggcaacttgaagagcgtggcccaggagcctgggccaccctgcggcctggggctggggctgggccttgggctgtgcggcaagcagggcccagccaccccggcccctgagcccagccgggccccagcatccctactcccaccagcgccagaacacagccccccgagctccccgctaacccagcccccagaggg

(19) United States
(12) Patent Application Publication
(10) Pub. No.: US 2003/0087867 A1
(43) Pub. Date: May 8, 2003

(54) Gene therapy for enhancing and/or inducing angiogenesis

(76) Inventors: Ronald V. Vogels, Linschoten (NL); Stefan F. F. Verlinden, Leiden (NL)

Correspondence Address: TRASK BRITT, P.O. BOX 2550, SALT LAKE CITY, UT, 84110, US

(21) Appl. No.: 10/224,249

(22) Filed: Aug. 19, 2002

Related U.S. Application Data
(63) Continuation of application No. 10/042,770, filed on Jan. 9, 2002.
Application No. 10/042,770 is a continuation of application No. PCT/NL00/00482, filed on Jul. 7, 2000.
(60) Provisional application No. 60/143,101, filed on Jul. 9, 1999.

(30) Foreign Application Priority Data
Jul. 9, 1999 (EP) 99202263.2

(51) Int. Cl. A61K 48/00; C12N 7/00; C12N 15/861; C12N 15/867

(52) U.S. Cl. 514/44; 424/93.2; 435/456; 435/235.1

(57) ABSTRACT

Gene therapy for enhancing and/or inducing angiogenesis and making a nucleic acid sequence encoding nitric oxide synthase (NOS). In particular, the nucleic acid sequence is administered in a systemic treatment, preferably comprising isolated tissue perfusion.

### CROSS-REFERENCE TO RELATED APPLICATION

This application is a continuation of application Ser. No. 10/042,770, filed Jan. 9, 2002, pending, which is a continuation of International Application Number PCT/NL00/00482 filed on Jul. 7, 2000 designating the United States of America, International Publication No. WO 01/03728 (Jan. 18, 2001), the contents of the entirety of which are incorporated by this reference.

### TECHNICAL FIELD

The present invention relates to biotechnology, more particularly to gene therapy vehicles and methods of delivery to stimulate the formation of new blood vessels (“angiogenesis”) in subjects with endothelial dysfunction. Disclosed is the delivery of therapeutic genes through blood circulation.

### BACKGROUND

Atherosclerosis is the accumulation of fatty deposits (plaque) inside blood vessels, leading to the blocking of the blood flow. Arteries throughout the body may be affected. The fibrous plaque forms occlusive lesions, because of its size and protusion into the arterial lumen. The fibrous cap covering the plaque may rupture, leading to thrombus formation, resulting in further occlusion of the artery. When the lesion is located in the coronary artery, rupture leads to a myocardial infarction. When the blood flow in brain vessels is blocked by lesions, stroke may result. In the limbs, the process of arterial narrowing leads to ischemia, blocking of the vessel, and, finally, possibly to limb necrosis.

The early phases of atherosclerosis are characterized by endothelial dysfunction. Many therapies have been investigated to assess the possibility to reverse the endothelial dysfunction and to stimulate the formation of new blood vessels (angiogenesis). It has recently been established that nitric oxide (“NO”) plays an important role in this process. Vascular endothelial dysfunction is characterized by the reduced release of NO in the arterial wall, which may cause a decrease in the blood flow in the arteries. Either process can lead to critical ischemia in the tissue drained by the affected vessel. Patients suffering from the consequences of endothelial dysfunction could benefit from therapies to increase new collateral blood vessel formation.

It is known that angiogenesis is mediated by a multitude of cytokines (like TNF-α and E-selectin) and angiogenic factors including bFGF (basic Fibroblast Growth Factor), VEGF (Vascular Endothelial Growth Factor), and TGF-β. Both bFGF and VEGF are key regulators of angiogenesis in adult tissues. They selectively stimulate proliferation of endothelial cells, starting with the binding of these growth factors to receptors present on the endothelial cell surface. Nitric oxide (NO) has been shown to play a role in this process. NO, originally identified as endothelium-derived relaxing factor, is an important endothelial vasoactive factor.

While both NO and angiogenic factors like bFGF and VEGF play a key role in the endothelial functions, their precise mode of action is not known. On the one hand, levels of angiogenic factors like bFGF and VEGF are increased in patients suffering from endothelial dysfunction. On the other hand, the release of nitric oxide in dysfunctional vascular endothelium is often reduced. This reduced release may cause constriction of the coronary arteries and thus contribute to heart disease. It is postulated that patients suffering from endothelial dysfunction could benefit from therapies to increase new collateral blood vessel formation and/or therapies to increase vasodilation.

Many experimental gene therapies concentrate on the stimulation of angiogenesis, in patients suffering from endothelial dysfunction, through the addition of VEGF or bFGF. Though these experimental therapies may have some effect, the level of therapy-induced angiogenesis is low, leading to a slow, if at all, recovery or enhancement of blood flow.

It has been demonstrated that NO is involved in VEGF-mediated proliferation of endothelial cells. Exposure of endothelial cells to VEGF was shown to lead to the activation of constitutive NO synthase (ceNOS, also called eNOS or NOSIII) and the release of biologically active NO. The proliferation of cells by VEGF can be inhibited by specific NOS-inhibitors like L-NAME, indicating that NO is an essential mediator in the VEGF-induced cell proliferation and angiogenesis.

Likewise, the presence of bFGF can increase ceNOS protein levels and enzyme activity during healing of rat gastric ulcers. Here also, the healing was inhibited specifically by the NOS-inhibitor L-NAME. In transgenic mouse models, disruption of the endogenous ceNOS gene impaired angiogenesis (Murohara et al.). This could not be compensated by the administration of VEGF, showing the essential role for NO in growth factor-mediated angiogenesis.

The art teaches that a decreased NO synthesis in endothelial cells may limit new blood vessel formation in patients with endothelial dysfunction. It has been suggested that oral L-arginine supplementation in the diet may be a therapeutic strategy to improve angiogenesis in patients with endothelial dysfunction. Recent data in animal studies show that activation of the NO-pathway may actually lead to a regression of the pre-existing intimal lesions in atherosclerosis.

### SUMMARY OF THE INVENTION

It is an object of the present invention to provide a gene therapy for local administration of NO in blood vessels and surrounding tissues. It is also an object of the invention to induce angiogenesis in patients with endothelial dysfunction and to provide gene therapy methods for the treatment of atherosclerosis.

It has been found that vectors expressing at least a gene encoding Nitric Oxide Synthase (NOS), either alone or in combination with genes encoding angiogenic factors, can be used to transfect cells in ischemic areas to enhance angiogenesis in limbs, and thus restore blood flow. The synthesis of NO is regulated by a family of isozymes. Three isoforms are known: nNOS, ceNOS and iNOS. Both nNOS and ceNOS are constitutively expressed and tightly regulated by calmodulin, whereas iNOS is induced by the action of cytokines. In the context of the present invention, the term Nitric Oxide Synthase (NOS) is intended to encompass all members of the mentioned isozyme family.

In one aspect, the invention provides a method for increasing NO and/or endothelial growth factors such as, but not limited to, VEGF and/or bFGF. In another aspect, the invention provides a method for increasing vasodilation of blood vessels. In yet another aspect, the invention provides a method for increasing angiogenesis through locally delivering an expression vector, preferably an adenovirus vector, comprising at least a nucleic acid encoding NOS, to sites selected for being provided with the capacity to induce, or at least in part promote, angiogenesis. Preferably, the delivery transpires through isolated tissue perfusion.

### BRIEF DESCRIPTION OF THE DRAWINGS

FIG. 1: Schematic representation of the adapter plasmid pAd5/CLIP.

FIG. 2: Schematic representation of the adapter plasmid Pad5/L420-HSApac.

FIG. 3: Schematic representation of the adapter plasmid pAdApt.

FIG. 4: Schematic representation of the adapter plasmid PAdApt-ceNOS.

FIG. 5: Relative blood flow after isolated limb perfusion in rat hind limb after occlusion of the artery and vena femoralis. Animals were treated with either isolated limb perfusion alone or isolated limb perfusion and delivery of 5×10⁹ Ad.CLIP.ceNOS infectious adenoviral particles. Blood flow was determined by Laser Doppler measurement of both footsoles. Using these measurements, the relative blood flow was calculated by dividing the amount of blood flow in the treated legs by the blood flow of the untreated leg. Measurements were performed directly before and after the procedure and from thereon every 3-4 days until the relative blood flow returned approximately to 1.

FIG. 6. Nucleotide sequence of cloned NOS cDNAs (SEQ ID NOS:13-16).

### DETAILED DESCRIPTION OF THE INVENTION

The present invention discloses the delivery of gene therapy vectors to a mammal carrying at least an NOS gene for use in enhancing vascularization/blood flow in stenosed limbs after isolated limb perfusion (ILP). Because in most applications of the invention the mammal is a human, it is in most applications of the invention, preferred that the nucleic acid molecule is a functional derivative from, or includes at least a functional fragment of, a nucleic acid molecule isolated from a human. The terms “functional derivative” and “functional fragment” are used here to indicate that the nucleic acid molecule encodes a peptide molecule with the same biological activity in kind, but not necessarily in amount, as NOS.

In gene therapy a molecule carrying genetic information is introduced in some or all cells of a host, whereby the genetic information is added to the host in a functional format.

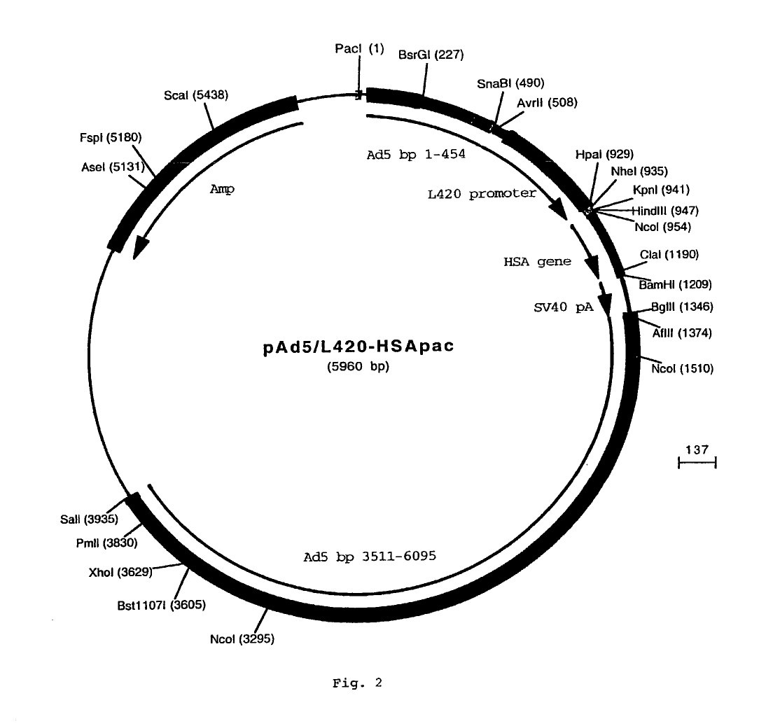

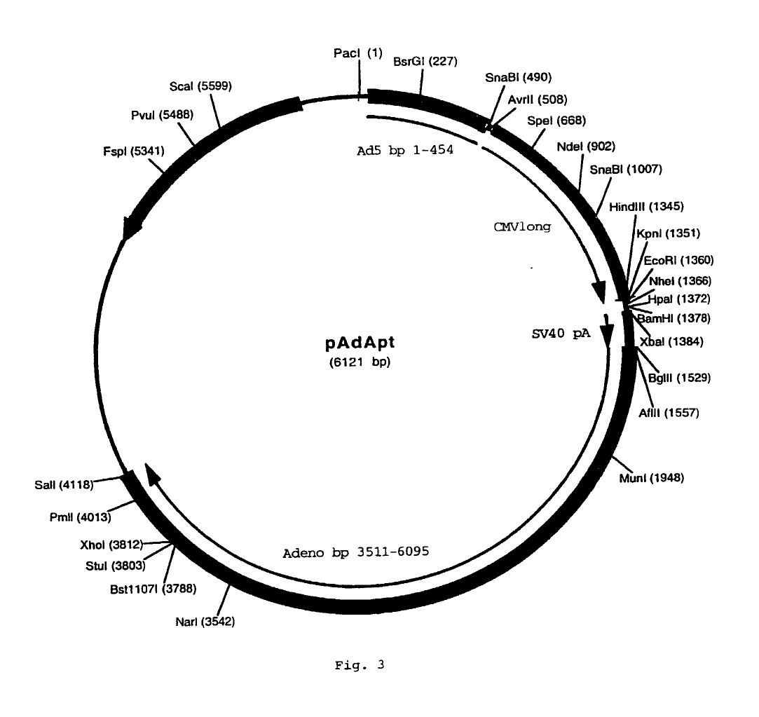

Three different isoforms of NOS have been identified. Two constitutively expressed isoforms are known, nNOS or NOSI, present in brain (Nakane et al., 1993), and ceNOS or NOSIII, present in endothelial cells (Janssens et al., 1992). Both isoforms are dependent on calmodulin and Ca₂+ for their activity. The third isoform (iNOS or NOSII, see Geller et al., 1993) is Ca₂+ independent, and its gene is induced by inflammation, microbial products and cytokines. Many cDNAs have been cloned and sequenced from different species and different tissues (see also FIG. 2). The isoforms share 50-60% sequence homology (for a recent review see Hobbs et al., 1999).

For the purpose of gene therapy, nucleic acid delivery vehicles are commonly used to introduce foreign genetic information into target cells. Suitable nucleic acid delivery vehicles for the present invention are those nucleic acid delivery vehicles capable of delivering nucleic acid to cells in vivo. Nonlimiting examples of such nucleic acid delivery vehicles are viral vectors, nonviral nucleic acid delivery vehicles and hybrids of viral and nonviral vehicles. Nonlimiting examples of suitable viral vectors are adenovirus vectors, adeno-associated virus vectors and retroviral vectors. Nonlimiting examples of nonviral nucleic acid delivery vehicles are liposomes, polyphosphazenes, etc. In hybrid systems elements, from viruses such as nucleic acid and/or proteins or parts thereof are incorporated into nonviral nucleic acid delivery vehicles to render the latter more effective.

Gene-transfer vectors derived from adenoviruses receive a lot of attention in the field of gene therapy. Adenoviruses are convenient viruses for construction of vectors for gene therapy, because of their high efficacy compared to other systems to deliver DNA in most mammalian cell types. Vectors derived from human adenoviruses, in which at least the E1 region has been deleted and replaced by a gene-of-interest, have been used extensively for gene therapy experiments in the pre-clinical and clinical phase.

There are several ways to administer recombinant adenovirus vectors. The recombinant virus can be injected intramuscularly, or be administered through a subcutaneous or an intravenous injection. These methods of administration have a disadvantage in that leakage of the vector from the site of injection into the blood system leads to a diffuse uptake in other organs, especially the liver. Systemic delivery of adenovirus vectors has been hampered by the fact that this results mainly in uptake of the vectors by the liver see: Connely S. et al., “High level tissue specific expression of functional human factor VIII in mice”, Human Gene Ther. 7(2):183-195 (1996); Herz J., Gerard, R. D. “Adenovirus mediated transfer of low density lipoprotein receptor gene acutely accelerates cholesterol clearance in normal mice”, Proc. Natl. Acad. Sci. USA 90:2812-2816 (1993)]. Other organs are not transduced or only minimally transduced by the adenovirus vectors.

The invention discloses the administration of a nucleic acid delivery vehicle without systemic delivery. Herein, an improved way of accessing ischemic areas in the limbs is provided by delivering the vectors directly via the bloodstream. In accordance with the invention, it is achieved that NO production in the endothelium is increased, thereby removing a cause for endothelial dysfunction. Preferably, the nucleic acid delivery vehicle comprises a virus-like particle. Preferably, the virus-like particle is an adenovirus particle, an adeno-associated virus particle and/or a retrovirus particle.

In one embodiment of the invention, adenoviral vectors are employed to deliver these genes. In a further embodiment of the present invention, adenoviral vectors are provided that lack the early genes E1 and E2A. The recombinant adenoviral vectors according to the invention may be derived from any wild-type adenovirus serotype that allows the functional expression of NOS in smooth muscle cells and/or in endothelial cells in the body of a mammal after administration of the recombinant adenoviral vector to the circulation of the mammal. Specifically, when the induction angiogenesis is the aim of the treatment, it is preferred to use a nucleic acid delivery vehicle capable of delivering the vehicle to preferably the smooth muscle cells lining the vessel wall. In this way, angiogenesis-promoting substances are delivered to a region localized close to the cells responsive for the substances or the products thereof. Furthermore, dilution of the substances and/or the products thereof into the blood is at least in part avoided, since the endothelial lining will at least in part prevent this. Striated muscle is not a tissue normally expressing NO and is therefore disfavored as the target cell for at least some of the NO-based angiogenesis promotion applications.

The present invention is exemplified on the basis of adenovirus vectors but is not limited to adenovirus vectors. In the examples given infra to illustrate the present invention, the recombinant adenoviral vectors are derived from human adenovirus type 5. Typically, one would like to optimize the delivery of the nucleic acid defined supra, particularly to cells of the vessel wall, particularly endothelial cells and/or smooth muscle cells. For this reason, in one embodiment of the invention, the nucleic acid delivery vehicle comprises a fiber protein derived from an adenovirus of a different subgroup than subgroup C, the subgroup that adenovirus serotype 5 belongs to. Preferably, the different subgroup is subgroup B, although subgroups D and/or F are also suitable. Preferably, the adenovirus of subgroup B is adenovirus 16 or adenovirus 35. It is to be understood, however, that those skilled in the art will be able to apply other viral vectors, such as other recombinant adenoviral vectors, without departing from the invention. Methods for the construction of recombinant adenoviral vectors according to the invention and for their propagation on useful packaging cells have been described in patent applications EP 0 707 071 and WO 97/00326, incorporated herein by reference. Other examples of vectors and packaging systems useful in the invention include, but are not limited to, those given in patent applications WO 93/19191, WO 94/28152, WO 96/10642, and WO 97/04119.

The invention provides means and methods for isolated tissue perfusion, preferably isolated limb perfusion (ILP), in which the blood circulation of the limb is isolated from the circulation of the body. The circulation through the limb is maintained by a pump. We have perfused the limb with an E1-deleted recombinant adenovirus vector harboring a human ceNOS gene. Thus, in a preferred embodiment, the invention provides a systemic treatment which includes isolated tissue perfusion.

Tissue perfusion is intended to read on isolated tissues as well as organs and/or extremities or any combination thereof. Two approaches of isolated perfusion are provided, one in which cells in the isolated perfused tissue are target cells for the delivery of nucleic acid and one in which the target cells for the delivery of nucleic acid are not in the isolated perfused tissue. Organs or body parts which are liable to be damaged by the treatment or which are likely to influence the uptake of virus by the target cells or which need to be isolated for a different reason can be excluded from the system to which the adenoviral vector encoding NOS activity is provided. For instance, liver delivery is for some applications not preferred. For instance, with the current thymidine kinase suicide approaches, it is better to avoid any possibility of toxicity for liver cells, since there are indications in mice that this organ is more sensitive for the cell killing effects of the treatment than other tissues. In the other isolated perfusion route, the vector is delivered to the isolated part only. In one embodiment, delivery of nucleic acid through isolated tissue perfusion of the heart is provided. In another embodiment, delivery of nucleic acid through isolated tissue perfusion of the liver is provided. The organs may be isolated for the normal circulation and perfused through methods known in the art and can be combined with the means and methods of the invention to improve delivery of the nucleic acid of interest. It is preferred to deliver the vector in the form of a virus-like particle. This means that the vector is packed in a virus shell, preferably an adenovirus shell, an adeno-associated virus shell or a retrovirus shell.

The present invention furthermore provides a pharmaceutical composition that comprises the nucleic acid delivery vehicle defined supra in combination with a diluent that is not toxic to the recipient mammal at the dosage used and that retains sufficient stability of the infectivity of the nucleic acid delivery vehicle for a time long enough to allow uptake of the nucleic acid delivery vehicle into the muscle cells and/or endothelial cells after administration of the composition to the circulation of the recipient mammal. Preferably, the nucleic acid delivery vehicle comprises an adenovirus vector. A typical nonlimiting example of a diluent according to this aspect of the invention is an isotonic saline solution that is sterile and that is buffered at a physiological pH. Preferably, the diluent furthermore contains serum-substituting ingredients. In the examples given infra to illustrate the present invention, Haemaccel (Behring Pharma) is used as a suitable diluent. It is to be understood, however, that those skilled in the art will be able to apply other diluents without departing from the invention. For some applications of the invention, it is furthermore preferred that the pharmaceutical composition is oxygenated prior to administration. Optionally, the nucleic acid delivery vehicle (the recombinant adenoviral vector and/or virus) is prepared in lyophilized form. In the latter case, the nucleic acid delivery vehicle is suspended in solution to obtain the pharmaceutical composition before administering the pharmaceutical composition to the circulation of the recipient mammal. Typically, a pharmaceutical composition comprising one dose of the virus-like particle, defined supra, contains at least about 10⁶, preferably about 10⁸, infectious units (iu) of the virus-like particle of the invention, but in certain conditions, it is preferred that it contains at least about 10⁹, more preferred 10¹⁰, or even more preferred 10¹¹ iu. The amount of virus to be provided depends on many parameters. As disclosed herein only a very limited portion of the administered virus actually infects the target cells. This may be one reason to increase the amount of virus to be administered. Another important aspect is, of course, the amount of NOS activity expressed by a cell infected with one or more viruses. This, of course, depends on the cell, but also on the promoter that drives the expression and its interaction with cell components of the expression machinery, etc.

In another aspect, the invention provides a method to deliver the nucleic acid molecule that encodes NOS to smooth muscle cells and/or endothelial cells in the body of a mammal, whereby the adenoviral vector or pharmaceutical composition defined supra is administered to a site in the circulation of the mammal. “Circulation” is meant to include both the blood circulation and the lymphatic circulation. Thus, the administration is performed to any site in the body of the recipient mammal where the blood or lymph fluids of the mammal pass. To more accurately restore blood flow, administrations of the nucleic acid delivery vehicle are preferably performed in conducting-arteria (intra-arterial or intravenous), where it is further preferred that the administration is into an artery located upstream of the ischemic area. Preferably, rather than expanding the capillary vessel network, novel conducting vessels are generated. Typically, occlusion occurs in the conducting vessels; therefore, typically expanding the capillary vessel network will have, at best, a limited effect on blood flow. The delivery of the nucleic acid delivery vehicle to conducting vessels will, at least in part, allow the preferred generation of novel conduction vessels. There are several means to perform the administration to the circulation. One of the means is by injection using, e.g., a syringe, a catheter or another infusion system known in the art. Preferably, the injection is performed at a controlled infusion rate. A much preferred means to perform the administration to the circulation is by perfusion. Perfusion is a technique whereby the administered pharmaceutical composition is caused to pass through the circulation or through a part of the circulation. When the administration is performed by perfusion, it is furthermore preferred that the perfusion is done multiple times by creating a closed circuit and repassaging the pharmaceutical composition through the circulation or part of circulation. Typically, the causing to pass is done by using a pump device and perfusion is performed at a rate depending on the species of the mammal to which the pharmaceutical composition is being administered. For humans, the rate is often in the range of approximately 40-80 ml/min and the perfusion is continued for a period of 15-90 minutes, but depending on patient, type of vascular endothelial dysfunction, and location thereof. These parameters may vary. For short treatment times (approximately 5-30 minutes) with the adenoviral construct, an anoxic perfusion can be performed by those skilled in the art by using balloon catheters to make a closed circuit. No heart-lung machine is necessary.

For optimal delivery of the nucleic acid molecule that encodes a Nitric Oxide Synthase to the target cells, preferably smooth muscle cells and/or endothelial cells, it is furthermore preferred that the blood of the mammal is first essentially washed away from the closed circuit (e.g., by precirculation with the diluent of the pharmaceutical composition only) before the pharmaceutical composition is administered. Optionally, the blood that is washed away is collected and readministered at the end of the procedure. The perfusion liquid can be oxygenated if needed. Essentially washing the blood from the closed circuit allows, at least in part, removal of antibodies that may affect the transduction procedure. Such may be the cases when the blood contains, or is suspected of containing, neutralizing antibodies against the nucleic acid delivery vehicle. Surgical techniques for perfusion of parts of the circulation according to the present invention are under development and are already available for various specific parts of the circulation, such as, e.g., the liver (Fraker, D L et al., Circulatory shock, 44, p.45-50,1994), the lung (Progrebniak H W et al., Ann. Thorac. Surg.,57, p.1477-83, 1994), and the kidney (Veen van de A H et al., Eur. J. Surg. Oncol. 20, p.404-405, 1994). A typical nonlimiting example of a routine perfusion technique useful in the invention is isolated limb perfusion (ILP), where a closed circuit is created between the femoral artery and the femoral vein. Alternatively, essentially the same perfusion techniques can be employed in the invention to exclude the delivery of the nucleic acid molecule to a part or parts of the circulation. In this aspect of the invention, the part or parts of the circulation to which the delivery is unwanted are perfused with a diluent according to the invention while the pharmaceutical composition is administered to the circulation systemically (hence, outside the perfusion circulation). An important example of this embodiment of the invention is exclusion of the liver circulation from delivery of the nucleic acid molecule.

The invention will now be elucidated by the following, nonrestrictive examples.

### EXAMPLES

### Example 1

Plasmid-based System for Rapid RCA-free Generation of Recombinant Adenoviral Vectors

A. Construction of Adenovirus Clones

pBr/Ad.Bam-rITR (ECACC Deposit P970821212

In order to facilitate blunt-end cloning of the ITR sequences, wild-type human adenovirus type 5 (Ad5) DNA was treated with Klenow enzyme in the presence of excess dNTPs. After inactivation of the Klenow enzyme and purification byphenol/chloroform extraction followed by ethanol precipitation, the DNA was digested with BamHl. This DNA preparation was used without further purification in a ligation reaction with pBr322-derived vector DNA prepared as follows: pBr322 DNA was digested with EcoRV and BamHI, dephosphorylated by treatment with TSAP enzyme (Life Technologies) and purified on LMP agarose gel (SeaPlaque GTG). After transformation into competent E. coli DH5α (Life Techn.) and analysis of ampicillin-resistant colonies, one clone was selected that showed a digestion pattern as expected for an insert extending from the BamHI site in Ad5 to the right ITR. Sequence analysis of the cloning border at the right ITR revealed that the most 3′ G residue of the ITR was missing, and the remainder of the ITR was found to be correct. The missing G residue is complemented by the other ITR during replication.

pBr/Ad.Cla-Bam (ECACC Deposit P97082117)

wt Adeno type 5 DNA was digested with ClaI and BamHI, and the 20.6 kb fragment was isolated from gel by electro-elution. pBr322 was digested with the same enzymes and purified from agarose gel by Geneclean. Both fragments were ligated and transformed into competent DH5α. The resulting clone pBr/Ad.Cla-Bam was analyzed by restriction enzyme digestion and shown to contain an insert with adenovirus sequences from bp 919 to 21566.

pBr/Ad.AflII-Bam (ECACC Deposit P97082114)

Clone pBr/Ad.Cla-Bam was linearized with EcoRI (in pBr322) and partially digested with AflII. After heat inactivation of AflII for 20 minutes at 65° C., the fragment ends were filled in with Klenow enzyme. The DNA was then ligated to a blunt double-stranded oligo linker containing a PacI site (5′-AATTGTCTTAATTAACCGCTTAA-3′ (SEQ ID NO: 1)). This linker was made by annealing the following two oligonucleotides: 5′-AATTGTCTTAATTAACCGC-3′ (SEQ ID NO:2) and 5′-AATTGCGGTTAATTAAGAC-3′ (SEQ ID NO:3), followed by blunting with Klenow enzyme. After precipitation of the ligated DNA to change buffer, the ligations were digested with an excess PacI enzyme to remove concatameres of the oligo. The 22016 bp partial fragment containing Ad5 sequences from bp 3534 up to 21566 and the vector sequences were isolated in LMP agarose gel (SeaPlaque GTG), religated and transformed into competent DH5α. One clone that was found to contain the PacI site and that had retained the large adeno fragment was selected and sequenced at the 5′ end to verify correct insertion of the PacI linker in the (lost) AflII site.

pBr/Ad.Bam-rITRpac#2 (ECACC Deposit P97082120) and pBr/Ad.Bam-rITR#8 (ECACC Deposit P97082121)

To allow insertion of a PacI site near the ITR of Ad5 in clone pBr/Ad.Bam-rITR, about 190 nucleotides were removed between the ClaI site in the pBr322 backbone and the start of the ITR sequences. This was done as follows: pBr/Ad.Bam-rITR was digested with ClaI and treated with nuclease Bal31 for varying lengths of time (2′, 5′, 10′ and 15′). The extent of nucleotide removal was followed by separate reactions on pBr322 DNA (also digested at the ClaI site), using identical buffers and conditions. Bal31 enzyme was inactivated by incubation at 75° C. for 10 minutes, the DNA was precipitated and resuspended in a smaller volume TE buffer. To ensure blunt ends, DNAs were further treated with T4 DNA polymerase in the presence of excess dNTPs. After digestion of the (control) pBr322 DNA with SalI, satisfactory degradation (˜-150 bp) was observed in the samples treated for 10 minutes or 15 minutes. The 10 minutes- or 15 minutes-treated pBr/Ad.Bam-rITR samples were then ligated to the above-described blunted PacI linkers (see pBr/Ad.AflII-Bam). Ligations were purified by precipitation, digested with excess PacI and separated from the linkers on an LMP agarose gel. After religation, DNAs were transformed into competent DH5α and colonies were analyzed. Ten clones were selected that showed a deletion of approximately the desired length and these were further analyzed by T-track sequencing (T7 sequencing kit, Pharmacia Biotech). Two clones were found with the Pacl linker inserted just downstream of the rITR. After digestion with Pacl, clone #2 has 28 bp and clone #8 has 27 bp attached to the ITR.

pWE/Ad.AflII-rITR (ECACC Deposit P97082116)

Cosmid vector pWE15 (Clontech) was used to clone larger Ad5 inserts. First, a linker containing a unique PacI site was inserted in the EcoRI sites of pWE15, creating pWE.pac. To this end, the double-stranded PacI oligo as described for pBr/Ad.AflII-BamHI was used but now with its EcoRI protruding ends. The following fragments were then isolated by electro-elution from agarose gel:pWE.pac digested with PacI, pBr/AflII-Bam digested with PacI and BamHI and pBr/Ad.Bam-rITR#2 digested with BamHI and PacI. These fragments were listed together and packaged using λ phage packaging extracts (Stratagene) according to the manufacturer's protocol. After infection into host bacteria, colonies were grown on plates and analyzed for presence of the complete insert. pWE/Ad.AflII-rITR contains all adenovirus type 5 sequences, from bp 3534 (AflII site) up to and including the right ITR (missing the most 3′ G residue).

B. Construction of New Adapter Plasmids

Generation of Adapter Plasmid pAd/L420-HSApac

The absence of sequence overlap between the recombinant adenovirus and E1 sequences in the packaging cell line is essential for safe, RCA-free generation and propagation of new recombinant viruses. The adapter plasmid pMLPI.TK (described in WO 97/00326) is an example of an adapter plasmid designed for use in combination with improved packaging cell lines like PER.C6 (described in WO 97/00326 and U.S. Pat. No. 08/892,873). This plasmid was used as the starting material to make new adapter plasmids in which nucleic acid molecules comprising specific promoter and gene sequences can be easily exchanged.

First, a PCR fragment was generated from pZipΔMo+PyF101(N⁻) template DNA (described in PCT/NL96/00195) with the following primers: LTR-1: 5′-CTG TAC GTA CCA GTG CAC TGG CCT AGG CAT GGA AAA ATA CAT AAC TG-3′ (SEQ ID NO:4) and LTR-2: 5′-GCG GAT CCT TCG AAC CAT GGT AAG CTT GGT ACC GCT AGC GTT AAC CGG GCG ACT CAG TCA ATC G-3′ (SEQ ID NO:5). Pwo DNA polymerase (Boehringer Mannheim) was used according to manufacturer's protocol with the following temperature cycles: once 5′ at 95° C.; 3′ at 55° C.; and 1′ at 72° C., and 30 cycles of 1′ at 95° C., 1′ at 60° C., 1′ at 72° C., followed by once 10′ at 72° C. The PCR product was then digested with BamHI and ligated into pMLP 10 (Levrero et al., 1991) vector digested with PvuII and BamHI, thereby generating vector pLTR10. This vector contains adenoviral sequences from bp 1 up to bp 454 followed by a promoter consisting of a part of the Mo-MuLV LTR having its wild-type enhancer sequences replaced by the enhancer from a mutant polyoma virus (PyF101). The promoter fragment was designated L420. Next, the coding region of the murine HSA gene was inserted. pLTR10 was digested with BstBI followed by Klenow treatment and digestion with NcoI. The HSA gene was obtained by PCR amplification on pUC18-HSA (Kay et al., 1990) using the following primers: HSA1, 5′-GCG CCA CCA TGG GCA GAG CGA TGG TGG C-3′ (SEQ ID NO:6) and HSA2, 5′-GTT AGA TCT AAG CTT GTC GAC ATC GAT CTA CTA ACA GTA GAG ATG TAG AA-3′ (SEQ ID NO:7). The 269 bp amplified fragment was subcloned in a shuttle vector using the NcoI and BglII sites. Sequencing confirmed incorporation of the correct coding sequence of the HSA gene, but with an extra TAG insertion directly following the TAG stop codon. The coding region of the HSA gene, including the TAG duplication, was then excised as an NcoI(sticky)-SalI(blunt) fragment and cloned into the 3.5 kb NcoI(sticky)/BstBI(blunt) fragment from pLTR10, resulting in pLTR-HSA10.

Finally, pLTR-HSA10 was digested with EcoRI and BamHI, after which the fragment containing the left ITR, packaging signal, L420 promoter and HSA gene was inserted into vector pMLPI.TK digested with the same enzymes and thereby replacing the promoter and gene sequences. This resulted in the new adapter plasmid pAd/L420-HSA that contains convenient recognition sites for various restriction enzymes around the promoter and gene sequences.

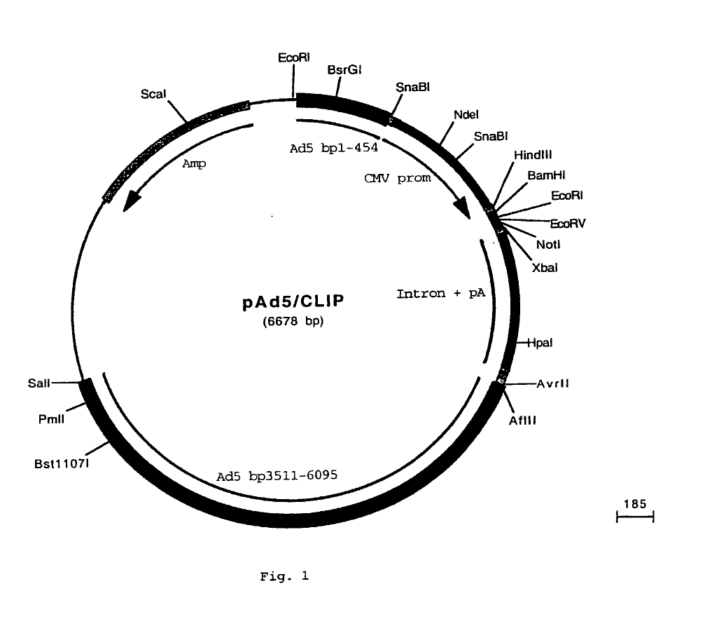

Another adapter plasmid that was designed to allow easy exchange of nucleic acid molecules was made by replacing the promoter, gene and poly A sequences in pAd/L420-HSA with the CMV promoter, a multiple cloning site, an intron and a poly-A signal. For this purpose, pAd/L420-HSA was digested with AvrII and BglII followed by treatment with Klenow to obtain blunt ends. The 5.1 kb fragment with pBr322 vector and adenoviral sequences was isolated and ligated to a blunt 1570 bp fragment from pcDNA1/amp (Invitrogen) obtained by digestion with HhaI and AvrII followed by treatment with T4 DNA polymerase. This adapter plasmid was named pAd5/CLIP (FIG. 1). To enable removal of vector sequences from the left ITR in pAd5/Clip, this plasmid was partially digested with EcoRI and the linear fragment was isolated. An oligo of the sequence 5′ TTAAGTCGAC-3′ (SEQ ID NO:8) was annealed to itself, resulting in a linker with an SalI site and EcoRI overhang. The linker was ligated to the partially digested pAd5/Clip vector and clones were selected that had the linker inserted in the EcoRI site 23 bp upstream of the left adenovirus ITR in pAd5/Clip, resulting in pAd5/Clipsal.

The vector pAd5/L420-HSA was then modified to create an SalI or PacI site upstream of the left ITR. Hereto, pAd5/L420-HSA was digested with EcoRI and ligated to a PacI linker (5′-AATTGTCTTAATTAACCGCTTAA-3′ (SEQ ID NO:1)). The ligation mixture was digested with PacI and religated after isolation of the linear DNA from agarose gel to remove concatamerized linkers. This resulted in adapter plasmid pAd5/L420-HSApac (FIG. 2).

Generation of Adapter Plasmids pAdMire and pAdApt

To create an adapter plasmid that only contains a polylinker sequence and no promoter or polyA sequences, pAd5/L420-HSApac was digested with AvrII and BglII. The vector fragment was ligated to a linker oligonucleotide digested with the same restriction enzymes. The linker was made by annealing oligos of the following sequence:

| | |
|---|---|
| PLL-1: | |
| 5′-GCC ATC CCT AGG AAG CTT GGT ACC | (SEQ ID NO:9) |
| GGT GAA TTC GCT AGC GTT AAC GGA TCC | |
| TCT AGA CGA GAT CTG G-3′ and | |
| PLL-2: | |
| 5′-CCA GAT CTC GTC TAG AGG ATC CGT | (SEQ ID NO:10) |
| TAA CGC TAG CGA ATT CAC CGG TAC CAA | |
| GCT TCC TAG GGA TGG C-3′. | |

The annealed linkers were digested with AvrII and BglII and separated from small ends by column purification (Qiaquick nucleotide removal kit) according to manufacterer's recommendations. The linker was then ligated to the AvrII/BglII-digested pAd5/L420-HSApac fragment. A clone, named pAdMire, was selected that had the linker incorporated and was sequenced to check the integrity of the insert.

Adapter Plasmid pAdMire Enables Easy Insertion of Complete Expression Cassettes.

An adapter plasmid containing the human CMV promoter that mediates high expression levels in human cells was constructed as follows: pAd5/L420-HSApac was digested with AvrII and 5′ protruding ends were filled in using Klenow enzyme. A second digestion with HindIII resulted in removal of the L420 promoter sequences. The vector fragment was isolated and ligated to a PCR fragment containing the CMV promoter sequence. This PCR fragment was obtained after amplification of CMV sequences from pCMVLacI (Stratagene) with the following primers:

| | |
|---|---|
| CMVplus: | |
| 5′-GATCGGTACCACTGCAGTGGTCAATATTGGCCA | (SEQ ID NO:11) |

TTAGCC-3′ and

| | |
|---|---|
| CMVminA: | |
| 5′-GATCAAGCTTCCAATGCACCGTTCCCGGC-3′. | (SEQ ID NO:12) |

The PCR fragment was first digested with PstI (underlined in CMVplus). after which the 3′-protruding ends were removed by treatment with T4 DNA polymerase. Then the DNA was digested with HindIII (underlined in CMVminA) and ligated into the above-described pAd5/L420-HSApac vector fragment digested with AvrII and HindIII. The resulting plasmid was named pAd5/CMV-HSApac. This plasmid was then digested with HindIII and BamHI and the vector fragment was isolated and ligated to the polylinker sequence obtained after digestion of pAdMire with HindIII and BglII. The resulting plasmid was named pAdApt (FIG. 3). Adapter plasmid pAdApt contains nucleotides −735 to +95 of the human CMV promoter (Boshart et al., 1985; A very strong enhancer is located upstream of an immediate early gene of human cytomegalovirus. Cell 41,521-530, 1985).

Generation of pAdApt-ceNOS

Plasmid pAC(d)CMVceNOS (described in Janssens et al. 1998; Human endothelial nitric oxide synthase gene transfer inhibits vascular smooth muscle cell proliferation and neointima formation after balloon injury in rats. Circulation 97, 1274-1281) was digested with EcoRI and the ends were filled in using Klenow enzyme. The ceNOS insert was then removed by digestion with XbaI and isolated from gel using the GeneClean kit II (Bio 101 Inc.). pAd/Clip was digested with BamHI and the ends were also filled in using Klenow followed by digestion with XbaI and isolation from gel.

Ligation of the Two Fragments Resulted in pAd/Clip-ceNOS.

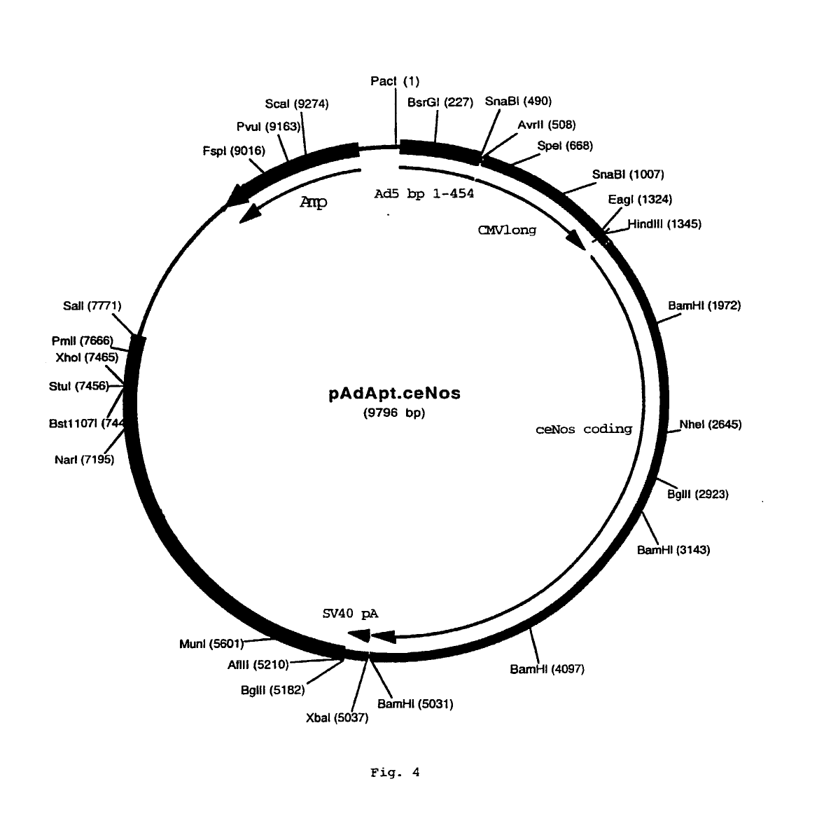

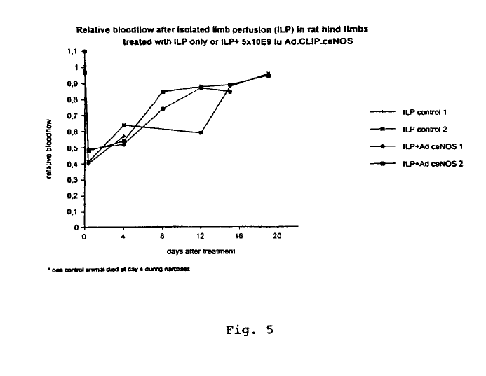

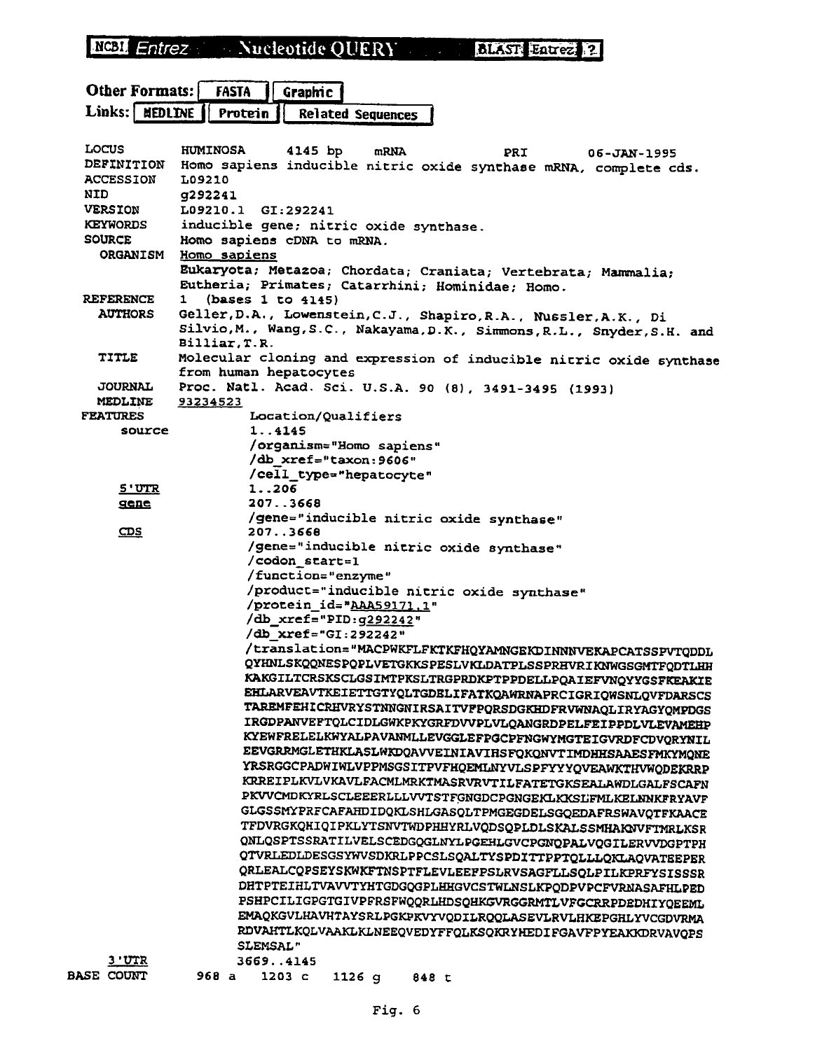

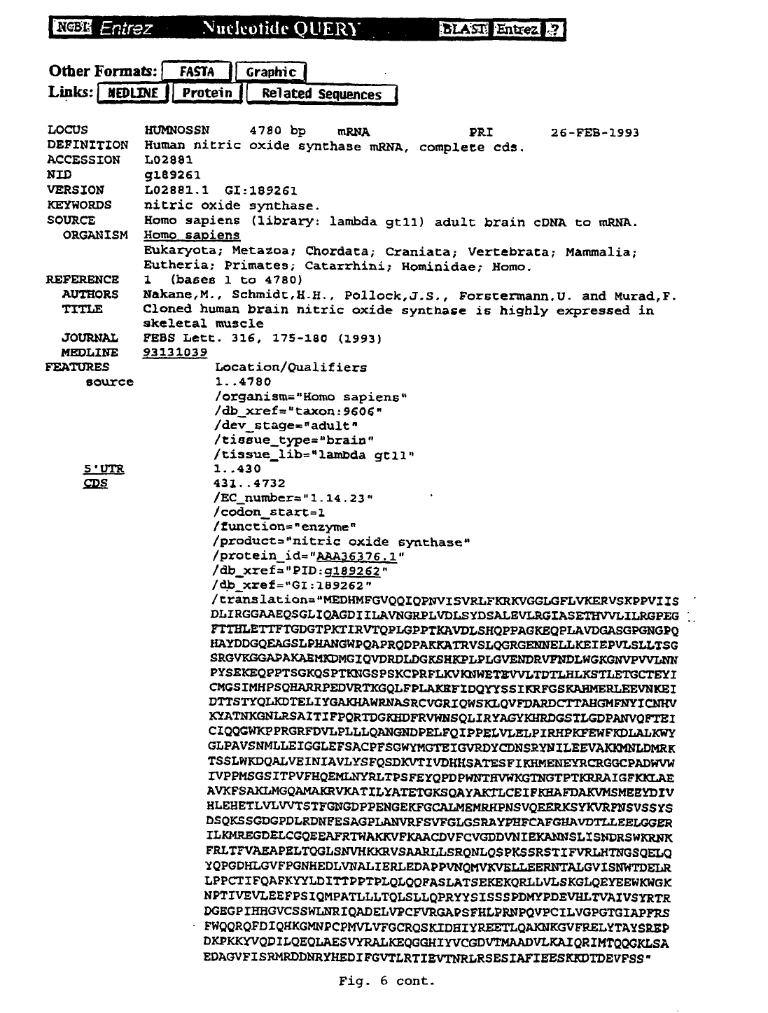

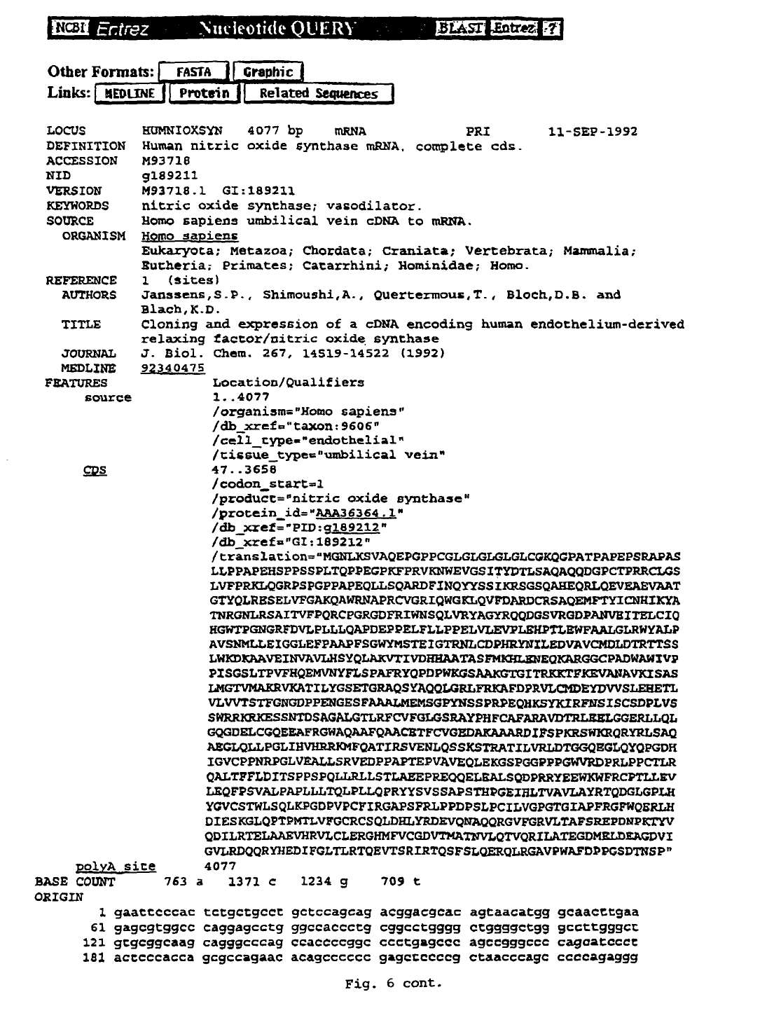

The ceNOS sequence was removed from pAdS/Clip-ceNOS by digestion with HindIII and XbaI and the 3.7 kb ceNOS fragment was isolated from gel using the GeneClean spinkit (Bio 101 Inc.) according to the manufacterer's instructions. Adapter plasmid pAdApt was also digested with HindIII and XbaI and the linear fragment was isolated as described above. Both fragments were ligated, resulting in pAdApt-ceNOS (FIG. 4).

The recombinant adenoviruses IGAdApt and IGAdApt-ceNOS were generated using the above-described adapter plasmids and the adenovirus cosmid clone pWE/Ad.AflII-rITR.

C. Generation of Recombinant Adenoviruses

E1-deleted Recombinant Adenoviruses

To generate E1-deleted recombinant adenoviruses with the new plasmid-based system, the following constructs were prepared: an adapter construct containing the expression cassette with the gene of interest linearized with a restriction enzyme that cuts at the 3′ side of the overlapping adenoviral genome fragment, preferably not containing any pBr322 vector sequences; and a complementing adenoviral genome construct pWE/Ad.AflII-rITR digested with PacI.

These two DNA molecules are further purified by phenol/chloroform and ETOH precipitation. Cotransfection of these plasmids into an adenovirus packaging cell line, preferably a cell line such as PER.C6 or a derivative thereof, generates recombinant replication-deficient adenoviruses by a one-step homologous recombination between the adapter and the complementing construct.

Recombinant adenovirus can be produced following introduction of the plasmids in the cell. It is to be understood that those skilled in the art may use other combinations of adapter and complementing plasmids without departing from the present invention.

A general protocol as outlined below and meant as a nonlimiting example of the present invention has been performed to produce several recombinant adenoviruses using various adapter plasmids and the Ad.AflII-rITR fragment. Adenovirus packaging cells (PER.C6) were seeded in ˜25 cm² flasks and, the next day, when they were at ˜80% confluency, were transfected with a mixture of DNA and lipofectamine agent (Life Techn.) as described by the manufacturer. Routinely, 40 μl lipofectamine, 4 μg adapter plasmid and 4 μg of the complementing adenovirus genome fragment AflII-rITR were used. Under these conditions, transient transfection efficiencies of ˜50% (48 hrs post-transfection) were obtained as determined with control transfections using a pAd/CMV-LacZ adapter. Two days later, cells were passaged to ˜80 cm² flasks and further cultured. Approximately five days later, a cytopathic effect (CPE) was seen, indicating that functional adenovirus has formed. Cells and medium are harvested upon full CPE and recombinant virus is released by freeze-thawing. An extra amplification step in a 80 cm² flask was routinely performed to increase the yield since, at the initial stage, the titers were found to be variable despite the occurrence of full CPE. After amplification, viruses were harvested and plaque purified on PER.C6 cells. Individual plaques were tested for viruses with active transgenes.

The recombinant adenovirus vectors were aliquoted to doses of 1×10¹⁰ iu (equal to approximately 10×10¹⁰ viral particles) and stored below −20° C. until use. Samples were thawed and kept at 4° C. until use.

Surgical and Perfusion Techniques

Surgical procedures were performed under Hypnorm anaesthesia (Janssen Pharmaceutica, Tilburg, The Netherlands). For isolated limb perfusion (ILP), a modification of the perfusion technique originally described by Brenckhuijsen was used. After an incision parallel to the inguinal ligament, the femoral artery and vein were approached and cannulated with silastic tubing (0.30 mm ID, 0.64 mm OD; 0.64 mm, 1.19 OD, respectively, Degania Silicone, Degania Bet, Israel). Collaterals were temporarily occluded by the application of a tourniquet around the groin, which was fixed to the inguinal ligament. An oxygenation reservoir and a roller pump (Masterflex) were included in the vascularly isolated circuit, which was, initially, perfused with haemaccel (Behring Pharma, Amsterdam, The Netherlands) for 3 minutes at a flow speed of 2 ml/min to wash out the blood. After the first wash-out step, recirculation was performed with recombinant adenoviruses (50 μl-1 ml) dissolved in 2.5-3.5 ml Haemaccel at the same flow rate for a time period of 15 minutes, followed by a second perfusion step of 5 minutes to wash out the nonbound virus with Haemaccel. During the perfusion and recirculation steps, the rat hind leg was kept at a constant temperature of 37-39°; a warm water mattress was applied around the leg. After the second wash-out step, the vascularly isolated circuit was discontinued and, after cannule removal, the femoral vessels were ligated. Previous experiments have shown that the collateral circulation via the internal iliac artery to the leg is so extensive that ligation of the femoral vessels can be performed without detrimental effects.

Revascularization Studies (NB: Pretreatment with Hemaccel to Remove IGG)

Revascularization was determined by measuring the blood flow in the legs by using a Laser Doppler apparatus.

Rats were used as an animal model, since rats have shown to be suitable for adenoviral vector testing. Rats were anaesthetized by using hypnorm. The blood flow in both hind limbs (the treated right and nontreated left legs) was measured by using a Laser Doppler apparatus according to the standard manufacturer's protocol.

Directly after ILP treatment, the blood flow in the hind legs was determined again by using the Laser Doppler apparatus.

Until day 30 after treatment, every 3 or 4 days the blood flow in the hind limbs was measured by using the Laser Doppler apparatus.

The method of isolated limb perfusion is used to deliver the recombinant adenoviral vectors since it has been shown in previous experiments to be able to deliver adenoviruses to the vasculature of the leg (see also FIG. 5). Furthermore ILP is a good model to study revascularization since the artery and vein used for virus delivery are disconnected from the blood circulation, causing severe ischemia in the manipulated limb.

### CITED LITERATURE

1. Nakane M, Schmidt H H, Pollock J S, Forstermann U, Murad F FEBS Lett Jan. 25, 1993; 316(2): 175-80 Cloned human brain nitric oxide synthase is highly expressed in skeletal muscle.

2. Janssens S P, Shimouchi A, Quertermous T, Bloch D B, Bloch K D J Biol Chem Jul. 25, 1992; 267(21):14519-22 Cloning and expression of a cDNA encoding human endothelium-derived relaxing factor/nitric oxide synthase.

3. Geller D A, Lowenstein C J, Shapiro R A, Nussler A K, Di Silvio M, Wang S C, Nakayama D K, Simmons R L, Snyder S H, Billiar T R Proc Natl Acad Sci U S A Apr. 15, 1993;90(8):3491-5 Molecular cloning and expression of inducible nitric oxide synthase from human hepatocytes.

4. Hobbs A J, Higgs A, Moncada S Annu Rev Pharmacol Toxicol 1999;39:191-220 Inhibition of nitric oxide synthase as a potential therapeutic target.

5. Kay, R., Takei, F., and Humphries, R. K. (1990). Expression cloning of a cDNA encoding M1/69. J. Immunol. 145, 1952-1959.

6. Levrero, M., Barban, V., Manteca, S., Ballay, A., Balsamo, C., Avantaggiata, M. L., Natoli, G., Skellekens, H., Tiollais, P., and Perricaudet, M. (1991). Defective and nondefective adenovirus vectors for expression of foreign genes in vitro and in vivo. Gene 101, 195-202.

What is claimed is:

1. A method of enhancing or inducing angiogenesis for essentially isolated tissue perfusion treatment, said method comprising:
administering to said essentially isolated tissue, a nucleic acid delivery vehicle comprising a nucleic acid encoding nitric oxide synthase (NOS) activity.

2. The method according to claim 1, wherein said perfusion treatment includes isolated limb perfusion.

3. The method according to claim 1, wherein said nucleic acid delivery vehicle comprises a virus-like particle.

4. The method according to claim 2, wherein said nucleic acid delivery vehicle comprises a virus-like particle.

5. The method according to claim 3, wherein said virus-like particle is selected from the group consisting of an adenovirus particle, an adeno-associated virus particle, and a retrovirus particle.

6. The method according to claim 4, wherein said virus-like particle is selected from the group consisting of an adenovirus particle, an adeno-associated virus particle, and a retrovirus particle.

7. A method of enhancing and/or inducing angiogenesis in a tissue systemically comprising:
isolating the tissue; and
perfusing said tissue with a nucleic acid delivery vehicle comprising a nucleic acid encoding nitric oxide synthase (NOS) activity.

8. A pharmaceutical composition for enhancing and/or inducing angiogenesis, said pharmaceutical composition comprising a nucleic acid encoding nitric oxide synthase (NOS) activity.

9. The pharmaceutical composition of claim 8, wherein said pharmaceutical composition further comprises a recombinant adenoviral vector encoding NOS activity.

10. The pharmaceutical composition of claim 8, wherein said pharmaceutical composition is a perfusion fluid.

11. The pharmaceutical composition of claim 9, wherein said pharmaceutical composition is a perfusion fluid.

12. The pharmaceutical composition of claim 9, wherein said perfusion fluid includes a virus-like particle comprising said recombinant adenoviral vector.

13. The pharmaceutical composition of claim 1 1, wherein said perfusion fluid includes a virus-like particle comprising the recombinant adenoviral vector.

14. The pharmaceutical composition of claim 12, wherein said virus-like particle is present in the pharmaceutical composition in an amount of from about 10⁶ to about 5.10⁹ iu.

15. The pharmaceutical composition of claim 13, wherein said virus-like particle is present in the pharmaceutical composition in an amount of from about 10⁶ to about 5.10⁹ iu.

16. A perfusion fluid for enhancing and/or inducing angiogenesis, said perfusion fluid comprising NOS activity provided by a nucleic acid delivery vehicle comprising nucleic acid encoding such NOS activity.

17. The perfusion fluid of claim 16, wherein said nucleic acid delivery vehicle comprises a virus-like particle.

18. The perfusion fluid of claim 17, wherein said virus-like particle is present in an amount of from about 10⁶ to about 5.10⁹ iu.

19. The perfusion fluid of claim 17, wherein said virus-like particle is present in an amount of from about 10⁶ to about 5.10⁹ iu.

20. The perfusion fluid of claim 17, wherein said virus-like particle is selected from the group consisting of a recombinant adenovirus particle, a recombinant adeno-associated virus particle, a recombinant retroviral particle, or a mixture of said particles.

21. A kit of parts for treatment to enhance and/or induce angiogenesis in a tissue, said kit of parts comprising:
the perfusion fluid of claim 16;
means for isolating tissues; and
means for perfusing said isolated tissues with said perfusion fluid.
